SEQUENCE LISTING (3 sequences)

SEQ ID NO: 1 - DNA, 18 bp, Artificial sequence
  Synthetic
cgggcgccactgctagag

SEQ ID NO: 2 - RNA, 18 nt, Artificial sequence
  Synthetic
gcccgcggugacgaucuc

SEQ ID NO: 3 - DNA, 18 bp, Artificial sequence
  Synthetic
gcccgcggtgacgatctc

(19) United States
(12) Patent Application Publication
(10) Pub. No.: US 2005/0014708 A1
(43) Pub. Date: Jan. 20, 2005

(54) Inhibition of viral replication by targeting RNA-DNA complexes

(76) Inventors: Daniel S. Pilch, Highland Park, NJ (US); Tsai-Kun Li, Edison, NJ (US)

Correspondence Address: Licata & Tyrrell P.C., 66 E. Main Street, Marlton, NJ, 08053, US

(21) Appl. No.: 10/844,705

(22) Filed: May 12, 2004

Related U.S. Application Data
(63) Continuation of application No. 09/945,155, filed on Aug. 31, 2001.

(51) Int. Cl. A61K 31/704; A61K 48/00; C12Q 1/68

(52) U.S. Cl. 514/36; 514/44; 514/37; 514/39

(57) ABSTRACT

This invention relates to anti-viral agents that target RNase H substrates of reverse transcriptase and methods of using these agents to inhibit reverse transcriptase and viral replication. The invention also relates to methods for screening anti-viral agents capable of inhibiting the RNase activity of reverse transcriptase by targeting RNA-DNA hybrid substrates.

### FIELD OF THE INVENTION

The present invention relates to the field of inhibition of viral replication by disrupting reverse transcriptase activity.

### BACKGROUND OF THE INVENTION

A pathogenic virus is an obligate intracellular parasite capable of causing disease. Numerous classes of pathogenic viruses (e.g., HIV, adenoviruses, herpes) have RNA as their genetic material. In order for viruses to reproduce, they must infect a host organism and use the host organism to make new viruses. Thus, pathogenic viruses replicate in a host organism. A critical component of the replication cycle of many of these RNA viruses is the conversion of the viral RNA to DNA, a process termed reverse transcription. Reverse transcription is catalyzed by a virally encoded enzyme called reverse transcriptase (RT). Inhibiting the activity of RT represents an important mechanism by which viral reproduction can be disrupted.

The human immunodeficiency virus (HIV), for example, begins replication by binding to a host's cell surface and fusing to the cell membrane. HIV targets T-4 lymphocyte cells because T-4 cells have numerous CD4 receptors proteins that allow HIV to bind to the cell. The viral nucleoproteins are then released into the cell's cytoplasm and transported into the nucleus. In the nucleus, HIV uses the cellular transcription machinery and the reverse transcriptase enzyme to make a DNA copy of the viral RNA. The viral DNA is then integrated into the host chromosomal DNA to form a provirus. A provirus is the term for the genome of a virus when it is integrated into the host cell DNA. The viral DNA is hidden within the host organism's DNA. Expression of the provirus leads to the generation of viral RNA and regulatory and structural proteins that are assembled into a new virus. As a result, when the host organism tries to make new proteins, new viruses are made as well.

Reverse transcriptase (RT) plays a pivotal role in the replication of human immunodeficiency virus type I (HIV-1). As a result, this enzyme is a key target in AIDS chemotherapies. Larder, B. A., Inhibitors of HIV reverse transcriptase as antiviral agents and drug resistance, Reverse Transcriptase (eds. Skalka, A. M. & Goff, S. P.) 205-222 (Cold Spring Harbor Laboratory Press, New York, 1993); Richman, D. D., HIV chemotherapy Nature 410, 995-1001 (2001). RT functions as a RNA-DNA dependent DNA polymerase, generating the DNA copy of the viral RNA, and a ribonuclease H (RNase H), degrading the RNA component of the RNA-DNA hybrid molecules.

Reverse Transcriptase may be inhibited by nucleoside reverse transcriptase inhibitors, non-nucleoside reverse transcriptase inhibitors, and nucleotide inhibitors. Nucleoside reverse transcriptase inhibitors employ defective nucleotides to prevent viral replication. When the defective nucleotides are incorporated into the DNA copy of the viral RNA, the DNA copy is not built properly and as a result is unable to incorporate into the host organism's DNA. Thus, new virus cannot be produced. Non-nucleoside RT inhibitors target and bind to the RT. By binding to RT, the non-nucleoside RT inhibitors prevent RT from converting the viral RNA into DNA and thus inhibit viral replication. Nucleotide RT inhibitors function in a similar fashion to nucleoside RT inhibitors, except the nucleotide RT inhibitors do not require chemical activation in order to carry out its function.

To date, the majority of the RT inhibitors that are in clinical use target the RNA-DNA dependent DNA polymerase activity of the enzyme. Richman, D. D., HIV chemotherapy, Nature 410, 995-1001. (2001). However, the efficacy of these anti-HIV-1 agents has been compromised by the high mutation frequency of the RT polymerase domain, which results in drug resistant viral strains. Thus, a continuing need exists for new classes of inhibitors that can combat HIV and other pathogenic viruses.

RNA degradation via the RNase H activity of HIV-1 RT is also a critical component of the reverse transcription process. Schatz, O. et al., Inactivation of the RNase H domain of HIV-1 reverse transcriptase blocks viral infectivity. Oncogenesis and AIDS (ed. Papas, T.) 304-315 (Portfolio Publishing, Texas, 1990); Tisdale, M., Schulze, T., Larder, B. A. & Moelling, K., Mutations within the RNase H domain of HIV-1 reverse transcriptase abolish viral infectivity. J. Gen. Virol. 72, 59-66 (1991); Champoux, J. J., Roles of ribonuclease H in reverse transcription. Reverse Transcriptase (eds. Skalka, A. M. & Goff, S. P.) 103-117 (Cold Spring Harbor Laboratory Press, New York, 1993). Viruses containing RNase H-defective RT are noninfectious. Schatz, O. et al,. Inactivation of the RNase H domain of HIV-1 reverse transcriptase blocks viral infectivity, Oncogenesis and AIDS (ed. Papas, T.) 304-315 (Portfolio Publishing, Texas, 1990); Tisdale, M., et. al., Mutations within the RNase H domain of HIV-1 reverse transcriptase abolish viral infectivity J. Gen. Virol. 72, 59-66 (1991). As a result, the RNase H activity of reverse transcriptase is essential for viral replication. Thus, interfering with the RNase H activity of RT represents a promising vehicle for the inhibition of HIV-1 replication.

### SUMMARY OF THE INVENTION

The present invention relates to a novel approach for inhibiting the ribonuclease (RNase) activity of reverse transcriptase (RT) by targeting its RNA-DNA hybrid substrates rather than the RT itself The present invention further relates to methods for screening potential anti-viral agents that inhibit RT activity by targeting viral RNA-DNA hybrid substrates. The present invention also relates to anti-viral agents that target RNA-DNA hybrid substrates and methods for their use in the inhibition of viral replication.

The present invention provides for anti-viral agents that target viral RNase RNA-DNA hybrid substrates of RT. The term ‘anti-viral agent’ refers to one or more molecule(s) or ligand(s) that inhibit the RNase activity of RT by targeting RNase RNA-DNA hybrid substrates of RT and/or binding to RNA-DNA hybrid substrates. The anti-viral agents of the present invention are not targeting the RT enzyme itself. As a result, the anti-viral agents and methods disclosed herein will have a higher immunity to the mutational frequency of RT and will be less susceptible to the development of viral resistant strains. The anti-viral agents of this invention will also have a inherent specificity for viral nucleic acid rather than host nucleic acid since the anti-viral agents are targeted to viral nucleic acid structures which are not found in host cells.

In preferred embodiments the ligand substantially binds to the RNA portion of the RNA-DNA hybrid substrate. In further embodiments, the ligand preferentially binds A-form and/or A-like conformations of nucleic acids. Examples of ligands that preferentially bind A-form relative to B-form nucleic acid structures include aminoglycosides of the neomycin and kanamycin classes. Robinson, H. & Wang, A. H.-J., Neomycin, spermine and hexaamminecobalt(III) share common structural motifs in converting B-to A-DNA, Nucleic Acids Res. 24, 676-682 (1996); Chen, Q., Shafer, R. H. & Kuntz, I. D. Structure-based discovery of ligands targeted to the RNA double helix Biochemistry 36,11402-11407 (1997). In further embodiments the aminoglycosides include neomycin, kanamycin, paromomycin, tobramycin and ribostamycin.

The term ‘RNA-DNA hybrid’ refers to an RNA paired to a complementary DNA strand. Thus, an ‘RNase RNA-DNA hybrid substrate’ refers to the portion of the viral RNA-DNA hybrid that is bound by RT and is the location of RNase activity of RT. One of the structural characteristics of a RNase H RNA-DNA hybrid substrate is its adoption of A-like conformations. Saenger, W., Principles of Nucleic Acid Structure, 220-241 (Springer-Verlag, New York, 1984).

The present invention also provides methods for inhibiting RT by targeting the RNA-DNA hybrid substrates that serve as the substrates in the RNase processing events of reverse transcription. In one embodiment of the invention, RT is inhibited by the introduction of anti-viral agents that are targeted to the RNase RNA-DNA hybrid substrates of RT. The anti-viral agent binds to the RNase RNA-DNA hybrid substrate and prevents the RT from cleaving the RNA. As a result, the anti-viral agent inhibits the RNase activity of RT at that site and disrupts viral replication.

In a further aspect of the invention, the ratio of anti-viral agent to RNase RNA-DNA hybrid substrate is 1:1, preferably 3:1 and most preferably 5:1.

In a further aspect of the invention, the RT induced cleavage of the viral RNA is inhibited by 80% and most preferably 100%.

The present invention also provides for a method for screening potential anti-viral agents that inhibit RT by targeting viral RNase RNA-DNA hybrid substrates comprising the steps of mixing potential anti-viral agents with specific viral RNase RNA-DNA hybrid substrates, adding RT to the agent-substrate mixture, and analyzing the effect of RT on the RNA-DNA hybrid substrate of the agent-substrate mixture. Inhibition of RT activity indicates an anti-viral agent. The potential anti-viral agents preferably target the specific RNase RNA-DNA hybrid substrate in a sequence or structure specific manner. In preferred embodiments, a technique called high throughput screening is employed to select anti-viral agents that inhibit the RNase activity of RT from a large number of potential anti-viral agents.

In a related aspect of the invention, molecules that target specific viral RNA-DNA hybrid substrates may be designed and developed based on the results and analyses of the screening methods.

This invention also provides for kits that screen potential anti-viral agents for their ability to inhibit the RNase activity of RT by targeting viral RNA-DNA substrates.

### BRIEF DESCRIPTION OF THE DRAWINGS

FIG. 1: Neomycin- and kanamycin-class aminoglycosides inhibit RT-Induced RNase H cleavage of the 18C-18R duplex. FIG. 1a is a schematic representation of the 18C-18R duplex, indicating the RNA-DNA junction. RNA bases are presented in lower case, while DNA bases are presented in upper case. The arrows denote the sites of RT-induced RNase H cleavage, with the relative thickness of the arrows reflecting the relative extents of cleavage. The asterisk denotes the site of the ³²P label. FIG. 1b, is a denaturing polyacrylamide gel showing RT-induced RNase H cleavage of the 18C-18R duplex in the absence or presence of aminoglycosides. All the lanes contain RT except lane 1. The neomycin concentrations in lanes 3, 4, 5, and 6 are 2.5, 12.5, 62.5, and 312.5 μM, respectively. The concentrations of the indicated aminoglycosides in lanes 7-11 are 312.5 μM. The arrows indicate the RNase H cleavage products, which are detailed in the text. FIG. 1c is a quantitation of the extent of cleavage at the primary cleavage site for lanes 2-5. FIG. 1d is a quantitation of the extent of cleavage at the primary cleavage site for the indicated lanes.

FIG. 2: Inhibition of RT-induced RNase H cleavage of the 18C-18D duplex by neomycin-class aminoglycosides. FIG. 2a is a schematic representation of the 18C-18D duplex, indicating the RNA-DNA junction. RNA bases are presented in lower case, while DNA bases are presented in upper case. The arrow denotes the site of RT-induced RNase H cleavage. The asterisk denotes the site of the ³²P label. FIG. 2b is a denaturing polyacrylamide gel showing RT-induced RNase H cleavage of the 18C-18D duplex in the absence or presence of aminoglycosides. All the lanes contain RT except lanes 1 and 2. The neomycin concentrations in lanes 4, 5, 6, and 7 are 0.25, 0.5, 1.0, and 2.0 mM, respectively. The concentrations of the indicated aminoglycosides in lanes 8 and 9 are 2.0 mM. The arrow indicates the RNase H cleavage product, which is detailed in the text. The marker is a 8mer RNA oligonucleotide with the sequence 5′-cgggcgcc-3′. FIG. 2c is a quantitation of the extent of cleavage for the indicated lanes. FIG. 2d illustrates that inhibition of RT- induced RNase H cleavage by neomycin is competitive. Denaturing polyacrylamide gel showing RT-induced RNase H cleavage of the 18C-18D duplex in the absence or presence of neomycin as a function of RT concentration. The neomycin concentration in lanes 3-6 is 1.0 mM. The amount of RT in lanes 1, 2, 3, 4, 5, and 6 is 0, 0.1, 0.1, 0.3, 0.9, and 2.7 μg, respectively.

FIG. 3: UV melting profiles for the 18C-18R(a) and 18C-18D(b) duplexes (filled circles) and their complexes with either neomycin (filled squares) or paromomycin (open circles) at a drug to duplex ratio (r_{dup}) of 3.0. For clarity of presentation, the melting curves were normalized by subtraction of the upper and lower baselines to yield plots of fraction single strand (α) versus temperature.

FIG. 4: ITC profiles at 25° C. for the titration of either neomycin (a, b) or paromomycin (c, d) into a solution of either 18C-18R(a, c) or 18C-18D(b, d). Each heat burst curve is the result of a 5 μl injection of drug, with all seven injections resulting in a final drug to duplex ratio (r_{dup}) of either 0.83 (b, d) or 1.25 (a, c).

FIG. 5: Schematic representation of the reverse transcription process catalyzed by HIV RT. The five RNase H cleavage events are indicated by the numerals 1-5.

### DETAILED DESCRIPTION OF THE INVENTION

The anti-viral agents that target RNase RNA-DNA hybrid substrates and methods for their use described in this invention provide for the inhibition of viral replication by disrupting RNase activity of RT. The methods of this invention include the use of anti-viral agents, which target RNase RNA-DNA hybrid substrates and bind to them, to disrupt the RNase activity of RT, by preventing the RT from cleaving the RNA, and ultimately inhibit viral replication. The following are preferred embodiments of the invention and are not intended to limit the invention.

Disruption of the RNase H activity of RT inhibits viral replication. RNA-DNA hybrid duplexes serve as substrates for the RNase H activity of RT. In a preferred embodiment of the invention, a method for inhibiting RT by targeting the RNA-DNA hybrid substrates of RNase H activity is disclosed. More specifically, RNA-DNA hybrid substrates of the RNase H activity of RT were mixed with different anti-viral agents that target RNase RNA-DNA hybrid substrates. The anti-viral agents may target the RNA-DNA hybrid substrates in a sequence specific and/or structure specific manner. Then, RT was added to the mixture to initiate the cleavage reaction. After incubation, the reaction was stopped, the RNA-DNA hybrid substrates were denatured and the cleavage products were resolved in a polyacrylamide gel. Analysis of the gels revealed that the anti-viral agents that target RNA-DNA hybrid substrates of RNase H activity inhibit RT.

In a preferred embodiment of the invention, a method for screening potential anti-viral agents that inhibit the RNase H activity of RT by targeting RNase H RNA-DNA hybrid substrates is disclosed. This method for screening potential anti-viral agents comprises the steps of: mixing potential anti-viral agents with specific RNase H RNA-DNA hybrid substrates, adding RT to the agent-substrate mixture, measuring the cleavage products of RT, and analyzing the effect of the potential anti-viral agent on the RNase H activity of RT. The methods disclosed herein may. be employed to screen numerous molecules and will allow for the design and development of anti-viral agents that target specific RNase RNA-DNA hybrid substrates, inhibit the RNase activity of RT, and ultimately viral replication. In preferred embodiments, high throughput screening methods are employed to screen potential anti-viral agents for their effect on the RNase activity of RT. High throughput screening is a process by which large numbers or libraries of compounds, the test compounds, are tested for their effect (i.e. binding activity or biological activity) on or against target molecules. For example, the test compounds may inhibit a target enzyme, act as an agonist or antagonist for receptor mediated intracellular process, or act as a competitor to the target molecule for binding to a receptor. Advantageously, high throughput screening allows for large numbers of compounds to be screened rapidly and at the same time. A large number of potential anti-viral agents may be screened for their effect on the RNase activity of RT by adding the potential anti-viral agents to target RNase RNA-DNA hybrid substrate that is labeled near the site of cleavage, introducing RT to the mixture, incubating the mixture, stopping the cleavage reaction, denaturing the RNA-DNA hybrid substrate and resolving and analyzing the cleavage products to determine the effect of the potential anti-viral agents on the RNase activity of RT. The molecules or compounds that inhibit the RNase activity of RT, i.e. the compounds/molecules that prevent the target RNA-DNA hybrid from being cleaved by RT, are anti-viral agents that inhibit RT, and ultimately viral replication, by targeting viral RNase RNA-DNA hybrid substrates.

The invention also provides for kits for use in screening potential anti-viral agents that inhibit the RNase activity of RT by targeting viral RNase RNA-DNA hybrid substrates. The kits may include reverse transcriptase, the reaction mixture that contains the target viral RNase RNA-DNA hybrid substrate that is labeled, and instructions to practice the methods of this invention. The reaction mixture is preferably a solution that does not interfere with the binding of the potential anti-viral agents to the hybrid substrates and the cleavage reaction of RT. The kits may further include components for use in performing assays of the RNase activity of RT such as mixtures to stop the cleavage reaction, mixtures for denaturing the RNA-DNA hybrid substrates and polyacrylamide gels to resolve the cleavage products. The kit may also include instructions for practicing high throughput screening of potential anti-viral agents.

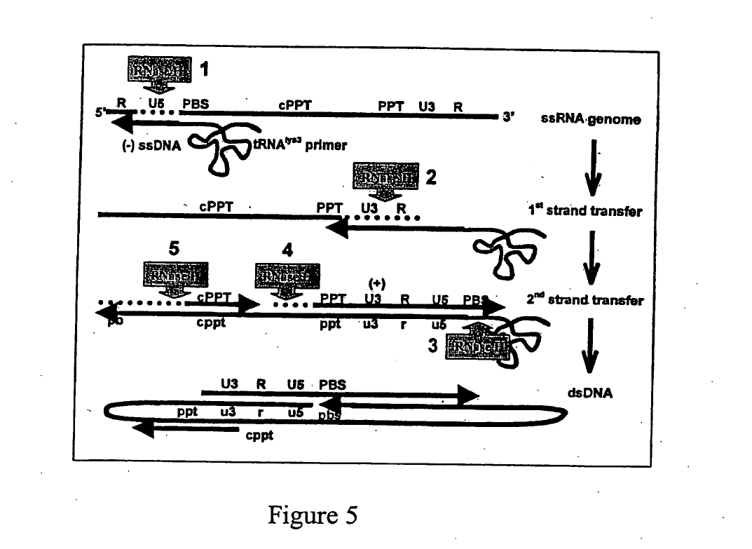

With respect to the HIV reverse transcription process, there are five different RNase H events, as depicted in FIG. 5. In principle, all five events may be inhibited by the same agent that targets RNase RNA-DNA hybrids. Thus, an anti-viral agent will be able to inhibit RT regardless of the stage of the reverse transcription process. In another embodiment of the invention, anti-viral agents that disrupt each of these events by targeting the specific RNase RNA-DNA hybrid substrate at the location where the specific event occurs may be determined using the screening methods disclosed herein. These screening methods are equally applicable in determining anti-viral agents for any virus that uses the reverse transcription process to replicate.

One of the structural characteristics of RNase H RNA-DNA hybrid duplexes is that they adopt A-like conformations. In a preferred embodiment of this invention, the RNase H activity of RT can be inhibited through the introduction of anti-viral agents which target the A-like conformations of RNase RNA-DNA hybrid duplexes to HIV infected cells. Agents that target and bind these A-like conformations disrupt RNase H activity and thereby, inhibit viral replication.

The impact of anti-viral agents, which target RNase H RNA-DNA hybrid substrates, on the RNase H activity of RT was evaluated through the use of model oligonucleotide duplexes that mimic two of the RNA-DNA hybrid substrates that are RNase H substrates of RT. One of the structural characteristics of such RNA-DNA hybrid duplexes is that they adopt A-like conformations. Aminoglycosides of the neomycin and kanamycin classes were employed as the anti-viral agents in this evaluation since they preferentially bind A-form relative to B-form nucleic acid structures were employed for this evaluation.

### EXAMPLE 1

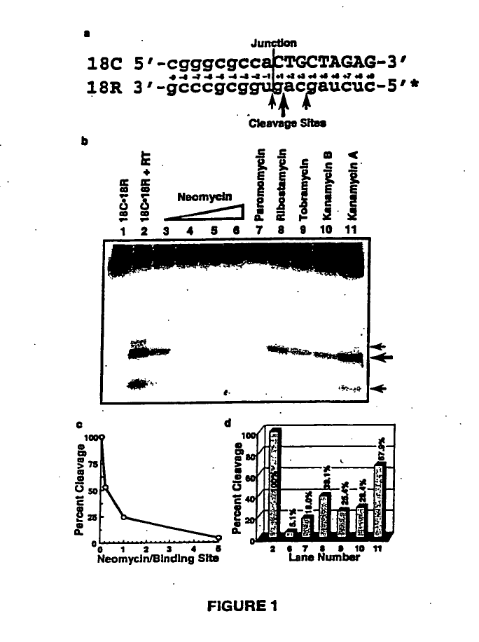

The first model oligonucleotide was a 18mer chimeric duplex (18C-18R). 18C-18R mimics a RNase H substrate during minus-strand strong-stop synthesis. (FIG. 1a). Mueller, U. et al. Crystal structure of an eight-base pair duplex containing the 3′-DNA RNA-5′ junction formed during initiation of minus-strand synthesis of HIV replication Biochemistry 37,12005-12011 (1998). Significantly, during the process of reverse transcription, this substrate is the first to be cleaved via the RNase H activity of RT. FIG. 1b shows the RT-induced RNase H cleavage profile of 18C-18R. Note that RT cleaves the RNA strand (18R) opposite the chimeric RNA-DNA strand (18C) at three sites (FIG. 1a, b). The primary cleavage site lies between base positions +1 and +2 (counting from the RNA-DNA junction in the 5′ direction on the-cleaved RNA strand). Two other minor cleavage sites lie between positions +3- and +4 and between positions −1 and +1, with the former site being cleaved to a greater extent than the latter site. Götte, M. et al., HIV-1 Reverse transcriptase-associated RNase H cleaves RNA/RNA in arrested complexes: Implications for the mechanism by which RNase H discriminates between RNA/RNA and RNA/DNA, EMBO J. 14, 833-841 (1995).

Recombinant HIV-1 RT p66 was isolated from Escherichia coli JM 109. Pandey, V. N. et al, Role of methionine 184 of human immunodeficiency virus type-1 reverse transcriptase in the polymerase function and fidelity of DNA synthesis, Biochemistry 35, 2168-2179 (1996). The 18C and 18R oligonucleotides were purchased from Dharmacon Research, Inc. (Lafayette, Colo.). The 18D oligonucleotide was obtained from Integrated DNA Technologies, Inc. (Coralville, Iowa.). Neomycin trisulfate trihydrate, the free base of tobramycin, paromomycin sulfate, and kanamycin B sulfate were obtained from Fluka, while kanamycin A monosulfate and ribostamycin sulfate dihydrate were obtained from Sigma

The RNase H activity of RT was assayed in a 10 μl reaction mixture containing 10 mM PIPES (pH 6.0) and 5 mM MgCl₂. Ten pmol of either 18C or 18R were ³²P-labeled at the 5′ end using T4 polynucleotide kinase. For the cleavage experiments, different aminoglycosides were mixed with 0.5 μM of target duplex. A small amount of RT, 0.1 μg, was then added to initiate the cleavage reaction. After incubation at 37° C. for 30 min, the reaction was stopped by addition of 3 M urea containing a mixture of bromophenol blue and xylene cyanol. Denaturation of the 18C-18R duplex required the addition of 5 M urea as well as heating at 95° C. for 5 min prior to gel loading. The cleavage products were resolved in a 10% polyacrylamide gel containing 8 M urea The gels were subsequently analyzed using a phosphoimager (Molecular Dynamics, Inc.).

Inspection of FIG. 1b reveals that both neomycin- and kanamycin-class aminoglycosides inhibit RT-induced cleavage of the 18R strand, with neomycin being the most potent inhibitor. At a stoichiometric concentration (corresponding to a neomycin to binding site ratio of 1), neomycin inhibits RT-induced cleavage at the primary site by 80%, achieving total inhibition at a neomycin to binding site ratio of 5 (FIG. 1c). Further inspection of FIG. 1b reveals that the extent to which the different aminoglycosides inhibit RT-induced cleavage follows the hierarchy:neomycin>paromomycin>tobramycin>kanamycin B>ribostamycin>>kanamycin A (see the quantitative analysis of the cleavage in FIG. 1d). This hierarchy of cleavage inhibition correlates with the corresponding hierarchy of ligand binding affinity for the 18C-18R duplex (discussed infra).

### EXAMPLE 2

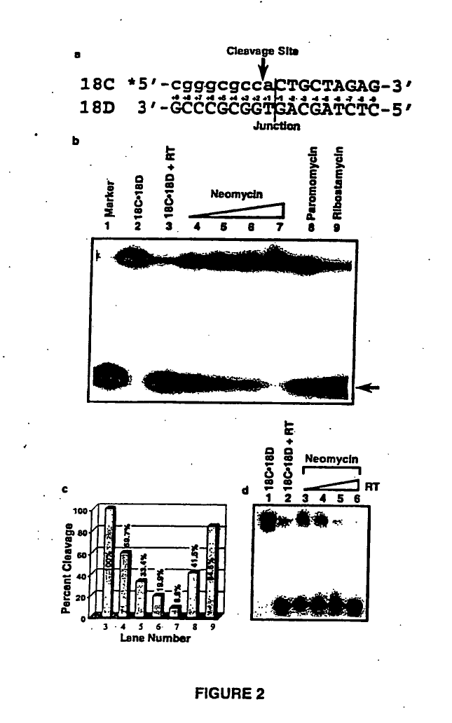

The second model oligonucleotide was a 18mer chimeric duplex (18C-18D). 18C-18D mimics a RNase H substrate during plus-strand strong-stop synthesis (FIG. 2a), which occurs following minus-strand DNA synthesis. Fedoroff, O. Y., et. al., B. R. Structural variation among retroviral primer-DNA junctions: Solution structure of the HIV-1 (−)-strand Okazali fragment r(gcca)d(CTGC)-d(GCAGTGGC), Biochemistry 35,11070-11080 (1996). For the 18C-18D duplex, RT cleaves the RNA portion of the chimeric strand (18C) opposite the DNA strand (18D) at only a single site (FIG. 2a , b), in marked contrast to the three cleavage sites observed in the 18C-18Rduplex. This unique cleavage site lies between base positions +1 and +2 (counting from the RNA-DNA junction in the 5‘direction on the cleaved chimeric strand). Furfine, E. S. & Reardon, J. E., Human immunodeficiency virus reverse transcriptase ribonuclease H: Specificity of tRNA(Lys3)-primer excision, Biochemistry 30, 7041-7046 (1991); Smith, J. S. & Roth, M. J. Specificity of human immunodeficiency virus-1 reverse transcriptase-associated ribonuclease H in removal of the minus-strand primer, tRNA(Lvs3), J Biol. Chem. 267, 15071-15079 (1992). Inspection of FIG. 2b reveals that neomycin-class aminoglycosides, inhibit RT-induced cleavage according to the hierarchy: neomycin>paromomycin>ribostamycin (see the quantitative analysis of the cleavage in FIG. 2c). As noted above for the 18C-18R duplex, this hierarchy of cleavage inhibition correlates with the relative binding affinities of the three ligands for the 18C-18D duplex. In addition to the neomycin class, the kanamycin class of aminoglycosides also inhibits RT-induced cleavage of the 18C-18D duplex.

Recombinant HIV-1 RT p66 was isolated from Escherichia coli JM 109. Pandey, V. N. et al, Role of methionine 184 of human immunodeficiency virus type-1 reverse transcriptase in the polymerase function and fidelity of DNA synthesis, Biochemistry 35, 2168-2179 (1996). The 18C and 18R oligonucleotides were purchased from Dharmacon Research, Inc. (Lafayette, Colo.). The 18D oligonucleotide was obtained from Integrated DNA Technologies’ Inc. (Coralville, Iowa). Neomycin trisulfate trihydrate, the free base of tobramycin, paromomycin sulfate, and kanamycin B sulfate were obtained from Fluka, while kanamycin A monosulfate and ribostamycin sulfate dihydrate were obtained from Sigma.

The RNase H activity of RT was assayed in a 10 μl reaction mixture containing 10 mM PIPES (pH 6.0) and 5 mM MgCl₂. Ten pmol of either 18C or 18R were ³²P-labeled at the 5′ end using T4. polynucleotide kinase. For the cleavage experiments, different aminoglycosides were mixed with 0.5 μM of target duplex. A small amount of RT, 0.1 μg, was then added to initiate the cleavage reaction. After incubation at 37° C. for 30 min, the reaction was stopped by addition of 3 M urea containing a mixture of bromophenol blue and xylene cyanol. Denaturation of the 18C-18R duplex required the addition of 5 M urea as well as heating at 95° C. for 5 min prior to gel loading. The cleavage products were resolved in a 10% polyacrylamide gel containing 8 M urea. The gels were subsequently analyzed using a phosphoimager (Molecular Dynamics, Inc.).

A comparison of FIGS. 1c and 2c reveals that approximately 420-times more neomycin is required to achieve 50% inhibition of RT-induced cleavage of the 18C-18D duplex relative to the 18C-18R duplex. As discussed below, one contributing factor to this observation is the higher affinity of neomycin for the 18C-18R duplex versus the 18C-18D duplex. However, the magnitude of the difference in neomycin binding affinity for the two duplexes (≈5-fold) is not sufficient to account for the 420-fold difference in inhibition of RT-induced cleavage. Another possible explanation for this difference in cleavage inhibition may lie in the location of the anti-viral agent binding site(s) on the host duplex relative to the site of cleavage. Both 18C-18R and 18C-18D have a nine base pair region of RNA-DNA, in which RT cleaves the RNA strand. However, the remaining nine base pair region in each of the two duplexes (from positions −1 to −9; FIG. 1a, 2a) differs with respect to strand composition. 18C-18R is RNA-RNA and 18C-18D is DNA-DNA. Duplex RNA is known to adopt an A-conformation, while duplex DNA adopts a B-conformation. Both neomycin- and kanamycin-class aminoglycosides are A-form specific nucleic acid binding agents. Hence, these drugs can target the RNA-RNA region of 18C-18R, but not the DNA-DNA region of 18C-18D. In fact, isothermal titration calorimetry (ITC) experiments reveal a neomycin binding stoichiometry of approximately five agents per duplex for 18C-18R and only three agents per duplex for 18C-18D. X-ray crystallographic studies by the Arnold group have demonstrated numerous contacts between the RNase H domain of RT and the base pairs between positions −1 and −9 (downstream from the cleavage site). Sarafianos, S. G. et al., Crystal structure of HIV-1 reverse transcriptase in complex with a polypurine tract RNA:DNA EMBO J. 20, 1449-1461 (2001). Comparatively few contacts were observed between RT and the base pairs between positions +1 and +9 (upstream from the cleavage site). The downstream RT-nucleic acid contacts are thought to be critical for the RNase H activity of RT. Sarafianos, S. G. et al., Crystal structure of HIV-1 reverse transcriptase in complex with a polypurine tract RNA:DNA, EMBO J. 20, 1449-1461 (2001). When 18C-18R serves as the RT substrate, neomycin can interfere with these important contacts by binding to the RNA-RNA region from base pair positions −1 to −9 (FIG. 1a). In contrast, when 18C-18D serves as the RT substrate, neomycin is unable to interfere with the downstream RT-nucleic acid contacts since it cannot target the DNA-DNA region of this duplex from base pair positions −1 to −9 (FIG. 2a). The differential ability of neomycin to target the −1 to −9 base pair regions of the 18C-18R and 18C-18D duplexes may account for the differential potency with which it inhibits RT-induced RNase H cleavage.

### EXAMPLE 3

To determine the nature of the aminoglycoside-induced inhibition of RT-catalyzed RNase H cleavage, the impact of RT concentration on neomycin-induced inhibition was examined. FIG. 2d shows the results of this determination using the 18C-18D duplex as the RT substrate. In the presence of 1 mM neomycin, increasing the concentrations of RT overcomes the RNase H inhibitory effect of the drug. This observation suggests that the manner in which neomycin inhibits the RNase H activity of RT is competitive in nature.

### EXAMPLE 4

The differential nucleic acid binding properties of the aminoglycosides were evaluated using spectrophotometric and calorimetric techniques. Neomycin and paromomycin are used as illustrative examples.

All UV absorbance experiments were conducted on an AVIV Model 14DS Spectrophotometer (Aviv Associates; Lakewood, N.J.) equipped with a thermoelectrically controlled cell holder. A quartz cell with a 1 cm pathlength was used for all the absorbance studies. Absorbance versus temperature profiles were measured at 274 nm with a 6 sec averaging time. The temperature was raised in 0.5° C. increments, and the samples were allowed to equilibrate for 1 min at each temperature setting. In these thermal denaturation studies, 18C-18R and 18C-18D solutions were 2 μM in duplex and contained aminoglycoside at concentrations ranging from 0 to 12 μM. The duplex solutions were preheated for S min at 85° C. and then cooled to room temperature prior to addition of the drug. The buffer solutions for the UV melting experiments contained 10 mM PIPES (pH 6.0) and 5 mM MgCl₂. For each optically detected transition, the melting temperature (Tₘ) was determined. Marky, L. A. & Breslauer, K. J. Calculating thermodynamic data for transitions of any molecularity from equilibrium melting curves, Biopolymers 26, 1601-1620 (1987).

Isothermal calorimetric measurements were performed at 25° C. on a MicroCal VP-ITC (MicroCal, Inc., Northampton, Mass.). In a typical experiment, 5 μl aliquots of 500 μM aminoglycoside were injected from a 250 μl rotating (430 RPM) syringe into an isothermal sample chamber containing 1.31 mL of either a 18C-18R solution that was 10 μM in duplex or a 18C-18D solution that was 15 μM in duplex. Each experiment of this type was accompanied by the corresponding control experiment in which 5 μL aliquots of 500 μM drug were injected into a solution of buffer alone. The duration of each injection was 5 seconds and the delay between injections was 240 seconds. The initial delay prior to the first injection was 60 seconds. Each injection generated a heat burst curve (μcal/sec vs. sec). The area under. each curve was determined by integration [using the Origin version 5.0 software (MicroCal, Inc., Northampton, Mass.] to obtain a measure of the heat associated with that injection. The heat associated with each drug-buffer injection was subtracted from the corresponding heat associated with each drug-duplex injection to yield the heat of drug binding for that injection. The calorimeter was calibrated both electronically and chemically. Pilch, D. S., Kirolos, M. A., Liu, X., Plum, G. E. & Breslauer, K. J. Berenil [1,3-bis-(4′-amidinophenyl)triazene]binding to DNA duplexes and to a RNA duplex: Evidence for both intercalative and minor groove binding properties. Biochemistry 34, 9962-9976 (1995). All duplex solutions were preheated for 5 minutes at 85° C. and then cooled to room temperature prior to their use in the ITC experiments. The buffer solutions for the ITC experiments contained 10 mM PIPES (pH 6.0) and 5 mM M_{g}Cl₂.

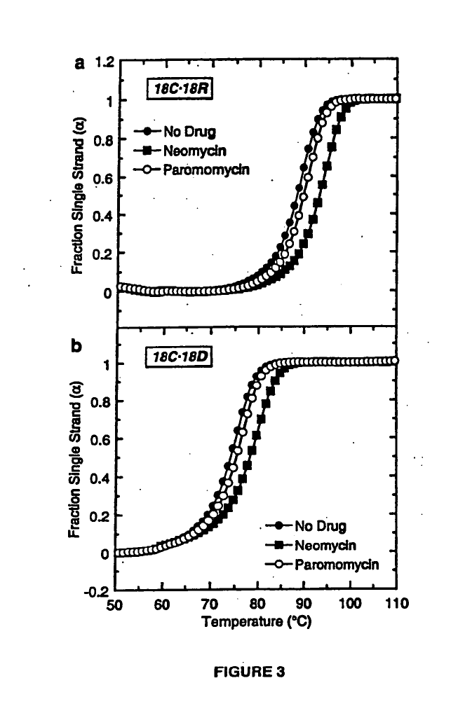

FIG. 3 shows the UV melting curves for the 18C-18R (FIG. 3a) and 18C-18D (FIG. 3b) duplexes in the absence and presence of neomycin or paromomycin at a drug to duplex ratio (r_{dup}) of 3.0. Note that the presence of either aminoglycoside enhances the thermal stability (Tₘ) of each target duplex, an observation consistent with each aminoglycoside binding to the two target duplexes with a preference for the duplex versus the single-stranded state. When 18C-18R serves as the target duplex, the binding of neomycin and paromomycin enhances the thermal stability of the duplex by 4.8 and 1.4° C., respectively (FIG. 3a). When 18C-18D serves as the target duplex, neomycin and paromomycin binding enhances the thermal stability of the duplex by 4.1 and 1.1° C., respectively (FIG. 3b). Thus, the extent of drug-induced enhancement in the thermal stabilities (ΔTₘ) of both target duplexes is greater for neomycin than for paromomycin. Furthermore, neomycin and paromomycin binding enhance the thermal stability of the 18C-18R duplex to a greater extent than the 18C-18D duplex. In other words, as measured by differences in ΔTₘ (ΔΔTₘ), both neomycin and paromomycin are able to distinguish between duplex targets that differ with respect to their strand composition (RNA versus DNA).

Higher r_{dup} ratios than 3.0 do not result in further increases in the Tₘ of the 18C-18D duplex, while yielding only marginal increases in the Tₘ of the 18C-18R duplex. The 18C-18R duplex has a nine base pair region of RNA-RNA from positions −1 to −9 and a nine base pair region of RNA-DNA from positions +1 to +9 (FIG. 1a). Neomycin- and kanamycin-class aminoglycosides bind to RNA-RNA duplexes with higher affinities than to the corresponding RNA-DNA hybrid duplexes. Thus, we assign the drug-induced increase in the Tₘ of the 18C-18R duplex at a r_{dup} ratio of 3.0 to drug complexation with the RNA-RNA region of the host duplex, while assigning the marginal increases in Tₘ observed at r_{dup}>3.0 to drug complexation with the RNA-DNA region of the duplex.

The ΔTₘ method described below was used to derive aminoglycoside-duplex association constants (Kₐ). Measured aminoglycoside-induced changes in the thermal stability of the host duplexes (FIG. 3) were used to estimate the apparent drug-duplex association constants at Tm (k_{T}_{ₘ}) from the following expression (Crothers, D. M. Statistical thermodynamics of nucleic acid melting transitions with coupled binding equilibria, Biopolymers 10, 2147-2160 (1971)):

$$\frac{1}{{T}_{m0}} - \frac{1}{{T}_{m}} = \frac{nR}{Δ {H}_{dup}} ln ( 1 + {K}_{{T}_{m}} L ) \qquad ( 1 )$$

In this expression, Tₘₒ and Tₘ are the melting temperatures of the drug-free and drug-bound duplex, respectively, n is the number of drug molecules bound per duplex, ΔH_{dup} is the enthalpy change for the melting of the duplex in the absence of bound drug, and L is the free drug concentration at Tₘ (which can be estimated by one half the total drug concentration). Values of ΔH_{dup} for the 18C-18R and 18C-18D duplexes were determined from the shapes of their UV melting profiles using the following relationship (Marky, L. A. & Breslauer, K. J. Calculating thermodynamic data for transitions of any molecularity from equilibrium melting curves, Biopolymers 26, 1601-1620 (1987)):

$$Δ {H}_{dup} = 6 {R ( {T}_{m} )}^{2} {( \frac{\partial α}{\partial T} )}_{T = {T}_{m}} \qquad ( 2 )$$

where α is the fraction of single strand. This calculation yielded ΔH_{dup} values of 157.8 kcal/mol for the 18C-18R duplex and 130.3 kcal/mol for the 18C-18D duplex. For meaningful comparisons, the calculated apparent binding constants at Tₘ should be extrapolated to a common reference temperature using the standard relationship:

$$\frac{\partial ln K}{\partial ( \frac{1}{T} )} = - \frac{Δ {H}_{b}}{R} \qquad ( 3 )$$

where ΔH_{b} is the enthalpy of drug binding.

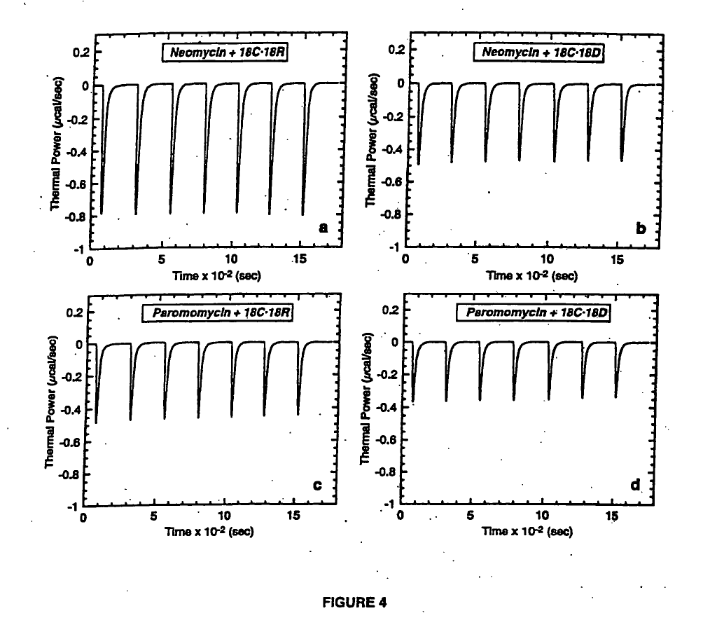

Table 1 summarizes the apparent. association constants at 25° C. that were calculated using equations (1)-(3) for neomycin and paromomycin binding to the 18C-18R and 18C-18D duplexes. The drug binding enthalpies (ΔH_{b}) required for extrapolation of the binding constants at Tₘ to common reference temperature of 25° C. using equation (3) were determined using isothermal titration calorimetry (ITC). FIG. 4 shows representative ITC profiles resulting from seven sequential injections of either neomycin (FIG. 4a, b) or paromomycin (FIG. 4c, d) into a solution of either 18C-18R (FIG. 4a, c) or 18C-18D (FIG. 4b, d). Each of the heat burst curves in FIG. 4 corresponds to a single drug injection, with the seven injections resulting in a final drug to duplex ratio (r_{dup}) of either 0.83 (when 18C-18D served as the target duplex) or 1.25 (when 18C-18R served as the target duplex). The areas under these heat burst curves were determined by integration to yield the associated injection heats. For each titration, the seven injection heats were similar in magnitude, an observation consistent with all of the injected drug being bound by the host duplex. These injection heats were corrected by subtraction of the corresponding dilution heats derived from the injection of identical amounts of drug into buffer alone. The resulting corrected injection heats then were divided by the total concentration of injected drug and averaged to yield the ΔH_{b} values listed in Table 1.

TABLE 1
ΔTₘ-derived association constants and
calorimetrically-derived enthalpies for the binding
of neomycin and paromomycin to the 18C-18R
and 18C-18D) duplexes at 25° C.

| | | Kₐ* | ΔH_{b}† |
|---|---|---|---|
| Duplex | Drug | (M⁻¹) | (kcal/mol) |
| 18C-18R | Neomycin | (8.6 ± 1.5) × 10⁶ | −8.8 ± 0.1 |
| 18C-18R | Paromomycin | (4.3 ± 1.6) × 10⁵ | −4.5 ± 0.1 |
| 18C-18D | Neomycin | (1.7 ± 0.3) × 10⁶ | −6.1 ± 0.1 |
| 18C-18D | Paromomycin | (1.9 ± 0.8) × 10⁵ | −3.6 ± 0.1 |

*Values of Kₐ were determined using the ΔTₘ analysis described in the text, with the indicated uncertainties reflecting the maximum errors in Tₘₒ, Tₘ, and ΔH_{b}, as propagated through equations (1) and (3).
†Binding enthalpies (ΔH_{b}) were determined by ITC as described in the text.

Inspection of the data in Table 1 reveals that the enthalpy of neomycin binding to the 18C-18R and 18C-18D duplexes is −8.8 and −6.1 kcal/mol, respectively, while the corresponding enthalpy of paromomycin binding to the two host duplexes is −4.5 and −3.6 kcal/mol, respectively. Thus, the binding of neomycin to the two target duplexes is enthalpically more favorable (exothermic) than the binding of paromomycin. Note the concordance between this ΔH_{b}-based trend and the ΔTₘ-based trend discussed above. A second feature that emerges from the ΔH_{b} data in Table 1 is that the binding of both neomycin and paromomycin to the 18C-18R duplex is enthalpically more favorable than the binding of these drugs to the 18C-18D duplex. Thus, both neomycin and paromomycin can enthalpically distinguish between the two target duplexes.

The data in Table 1 reveals that the affinity of neomycin for each of the host duplexes is greater than the corresponding affinity of paromomycin (a 20-fold difference in affinity for the 18C-18R duplex and a 9-fold difference in affinity for the 18C-18D duplex). The differential neomycin and paromomycin binding affinities correlate with the differential abilities of these drugs to inhibit RT-induced RNase H cleavage (compare the binding affinities in Table 1 with FIG. 1d, 2c). Similar correlations are obtained for the other neomycin- and kanamycin-class aminoglycosides. These correlations suggest that the inhibition of RT-induced RNase H cleavage by the aminoglycosides is dictated by their abilities to bind to the target nucleic acid duplexes.

Note that neomycin exhibits a 5-fold greater affinity for the 18C-18R duplex than for the 18C-18D duplex, while paromomycin exhibits a corresponding 2-fold greater affinity. These differential affinities of neomycin and paromomycin for the 18C-18R and 18C-18D duplexes contribute, at least in part, to the differential potencies with which these drugs inhibit RT-induced RNase H cleavage of the two target duplexes.

The above described examples demonstrate a novel approach for inhibiting HIV-1 RT by aminoglycoside targeting of the RNA-DNA structures that serve as substrates for the RNase H activity of the enzyme, an activity critical for viral replication. Neomycin has been shown to inhibit HIV-1 production in chronically infected cells. Zapp, M. L., Stem, S. & Green, M. R., Small molecules that selectively block RNA binding of HIV-1 Rev protein inhibit Rev function and viral production, Cell 74, 969-978 (1993). The basis for this anti-HIV-1 activity of neomycin has been attributed to its ability to interfere with the interactions between viral proteins and their specific RNA substrates (e.g., Rev/RRE and Tat/TAR). Zapp, M. L., Stem, S. & Green, M. R., Small molecules that selectively block RNA binding of HIV-1 Rev protein inhibit Rev function and viral production, Cell 74, 969-978 (1993); Wang, S., Huber, P. W., Cui, M., Czarnik, A. W. & Mei, H.-Y., Binding of neomycin to the TAR element of HIV-1 RNA induces dissociation of the Tat protein by an allosteric mechanism, Biochemistry 37, 5549-5557 (1998); Hamy, F. et al., A new class of HIV-1 Tat antagonist acting through Tat-TAR inhibition Biochemistry 37, 5086-5095 (1998); Mei, H.-Y. et al., Inhibitors of protein-RNA complexation that target the RNA: Specific recognition of human immunodeficiency virus type I TAR RNA by small organic molecules, Biochemistry 37, 14204-14212 (1998). These RNA substrates, as well as the RNA-DNA substrates discussed infra, all represent potential anti-HIV-1 targets for neomycin. However, the processing of the RNA-DNA substrates studied here via the RNase H activity of RT occurs earlier in the HIV-1 life cycle than the interactions of Tat and Rev with their RNA targets (Tang, H., Kuhen, K. L. & Wong-Staal, F. Lentivirus replication and regulation, Annu. Rev. Genet. 33, 133-170 (1999)), and may therefore be the neomycin targets that are primarily responsible for the anti-HIV-1 activity of the drug. In the aggregate, the data provides for new anti-HIV-1 agents that can target RT nucleic acid substrates in a sequence-specific and/or structure-specific manner. One skilled in the art will recognize that the methods disclosed herein are generally applicable to the inhibition of any virus that replicates via the process of reverse transcription.

The methods and procedures disclosed herein may be employed and/or adapted for use in screening potential anti-viral agents that target RNA-DNA substrates of RT.

One skilled in the art will readily appreciate that the anti-viral agents of the present invention may be adapted for use in the treatment of HIV in similar fashion to those anti-HIV drugs known in the art and prescribed to patients infected with HIV. The anti-viral agents may also be adapted for use in the treatment of patients infected with a virus that replicates using the process of reverse transcription.

One skilled in the art will readily appreciate that the present invention is well adapted to carry out the objects and obtain the ends and advantages mentioned as well as those inherent therein. The anti-viral agents along with the methods, procedures and treatments described herein are presently representative of preferred embodiments and are exemplary and not intended as limitations on the scope of the invention. Changes therein and other uses will occur to those skilled in the art which are encompassed within the spirit of the invention or defined by this scope with the claims.

It will be readily apparent to one skilled in the art that varying substitutions and modifications may be made to the invention disclosed herein within departing from the scope and spirit of the invention.

All patents and publications are herein incorporated by reference to the same extent as if each individual publication was specifically and individually indicated to be incorporated by reference.

1. An anti-viral agent comprising a ligand that targets RNase H RNA-DNA hybrid substrates of RT, wherein the ligand inhibits the RNase H activity of RT.

2. A method for inhibiting reverse transcriptase comprising the step of:
targeting RNA-DNA hybrid substrates of the RNase H activity of RT, wherein the anti-viral agent of claim 1 targets and binds to the RNA-DNA hybrid substrates, thereby inhibiting the RNase H activity of reverse tanscriptase.

3. The method of claim 2, wherein the anti-viral agent is an aminoglycoside.

4. The method of claim 3, wherein the aminoglycoside is selected from a group consisting of neomycin, kanamycin, paromomycin, tobramycin and ribostamycin.

5. The method of claim 2, wherein the anti-viral agent is neomycin.

6. The method of claim 5, wherein the neomycin to RNA-DNA hybrid substrate ratio is 1:1.

7. The method of claim 6, wherein the neomycin inhibits reverse transcriptase induced cleavage of the substrate by 80% at the primary site.

8. The method of claim 5, wherein the neomycin to RNA-DNA hybrid substrate ratio is 5:1.

9. The method of claim 8, wherein the neomycin completely inhibits reverse transcriptase induced cleavage of the substrate at the primary site.

10. An anti-HIV-1 agent comprising an aminoglycoside that targets RNase H RNA-DNA hybrid substrates of reverse transcriptase, wherein the aminoglycoside prevents the reverse transcriptase from cleaving the HIV-1 RNA, thereby inhibiting replication of HIV-1.

11. A method for inhibiting HIV-1 reverse transcriptase comprising the step of:
targeting RNase H RNA-DNA hybrid substrate of RT, wherein the anti-HIV-1 agent of claim 10 targets and binds to the RNA-DNA hybrid substrate at the location of RNase H activity, thereby inhibiting viral replication of HIV-1.

12. The method of claim 11, wherein the anti-HIV-1 agent is an aminoglycoside selected from a group consisting of neomycin, kanamycin, paromomycin, tobramycin and ribostamycin.

13. A method for screening potential anti-viral agents that inhibit RT activity by targeting RNase RNA-DNA substrates of RT comprising the steps of:
(a) mixing a potential anti-viral agent with a specific RNase RNA-DNA hybrid substrate,
(b) adding RT to the mixture in step (a),
(c) resolving the cleavage product of RT, and
(d) analyzing the effect of the potential anti-viral agent on the RNase activity of RT.

14. A high throughput screening method for identifying anti-viral agents that inhibit the RNase activity of RT by targeting RNase RNA-DNA hybrid substrates comprising the steps of:
(a) selecting a group of potential anti-viral agents,
(b) introducing each potential anti-viral agents from the group to a reaction mixture containing labeled target RNase RNA-DNA hybrid substrate,
(c) introducing RT to each mixture in step (b),
(d) resolving the cleavage products of RT for each mixture in step (c), and
(e) analyzing each potential anti-viral agent for its ability to inhibit the RNase activity of RT.

15. A kit for screening potential anti-viral agents that target RNase RNA-DNA hybrid substrates comprising:
(a) a reaction mixture containing a labeled target RNase RNA-DNA hybrid substrate,
(b) reverse transcriptase, and
(c) instructions for use.

16. The kit of claim 15, further comprising a mixture to stop the cleavage reaction of RT.

17. The kit of claim 16, further comprising a mixture for denaturing the RNA-DNA hybrid substrate.
